(19) United States
(12) Patent Application Publication
(10) Pub. No.: US 2016/0060639 A1
(43) Pub. Date: Mar. 3, 2016

(54) Positive-Selection Cloning and Expression Vector Based on the Toxicity of Killin

(71) Applicant: Peng Liang, Nashville, TN (US)

(72) Inventor: Peng Liang, Nashville, TN (US)

(21) Appl. No.: 14/468,805

(22) Filed: Aug. 26, 2014

(51) Int. Cl. C12N 15/72

(52) U.S. Cl. CPC C12N 15/72; C12N 2830/002; C12N 2500/34

(57) ABSTRACT

Here we report the creation of a new positive-selection cloning vector dubbed pKILLIN, which overcomes all of the above pitfalls. The essence behind its high cloning efficiency is the extreme toxicity and small size of the toxic domain of killin, a recently discovered p53 target gene. Insertion inactivation of killin within the multiple cloning site via either blunt- or sticky-end ligation serves not only as a highly efficient cloning trap, but also may allow any cloned genes to be expressed as His-tagged fusion proteins for subsequent purification. Thus, pKILLIN is a versatile positive-selection vector ideal for cloning PCR products, making DNA libraries, as well as routine cloning and bacterial expression of genes.

### BACKGROUND

Since its invention in the early 1970s, gene cloning has become a routine practice in modern biomedical laboratories. DNA fragments or PCR products are often cloned into a plasmid vector for subsequent sequence analysis, expression or other molecular biological manipulations. A number of strategies developed over the years made the screening of the recombinant plasmids more efficient. Blue/white screening is one of the most widely used approaches for this purpose. When transformed into an E. coli host harboring the lacZ ΔM15 allele and plated on a LB plate with 5-bromo-4-chloro-3-indolyl-β-D-galactopyranoside (X-gal), plasmids containing the lacZα gene produce blue colonies due to “α-complementation” of the truncated β-galactosidase encoded by lacZ ΔM15. White colonies are produced when a foreign DNA fragment is inserted into the lacZα gene, thereby disrupting its function. Various vectors employing this mechanism have been reported. However, the approach has the following drawbacks: false positives (white colonies without inserts) or false negative colonies (blue colonies with inserts); high background of blue colonies from vector self-ligation; substrates such as X-Gal and isopropyl-β-D-thiogalactopyranoside (IPTG) are necessary and costly; requirement for vector dephosphorylation to reduce colonies formed from self-ligation when a single restriction site is used; time needed for the color development and hosts must contain lacZ ΔM15 allele. The same drawbacks from the lacZ-Xgal system remain for vectors based on other color selection schemes employing different metabolic enzymes and substrates. Vectors using green fluorescent protein (GFP) gene as an indicator requires cells to be exposed to toxic UV light.

A cloning vector with positive selection is an alternative strategy that makes the selection of recombinant DNA simpler and more efficient. The approach often employs a plasmid with the expression of a lethal gene as an indicator to prevent growth of the host bacteria, whereas insertion of any foreign DNA fragments or PCR products disrupts the coding sequence of the toxic indicator gene, thus resulting in growth of colonies containing recombinant plasmids. A number of positive-selection cloning vectors have been described, including some that are commercially available. However, due to pitfalls listed below, positive-selection cloning vectors have yet to catch on, both in applications and popularity. These pitfalls include: high background resulting from either incomplete lethality of the indicator gene employed or the incomplete inactivation of the indicator gene due to its large size; limited cloning sites available for insert or limited unique flanking sites for retrieving DNA fragments cloned; requirement for special treatment or substrates on the LB agar plate in order to confer lethality of the indicator genes. Moreover, few positive-selection cloning vectors reported could allow simultaneous expression of the recombinant gene of interest with or without a tag to assist in subsequent purification of the target protein.

### SUMMARY

Here we describe a highly efficient positive-selection cloning vector dubbed pKILLIN. It is based on the toxicity of the killin gene we recently discovered, which alleviates most of the above pitfalls. Killin is a p53 target gene that encodes a high-affinity single-stranded DNA-binding protein required for S-phase arrest and apoptosis in mammalian cells. When expressed in E. coli, Killin is highly toxic and causes total lethality of the host cells. Systematic deletion analysis revealed that the lethality resided in the N-terminus of Killin, with only 42 amino acid residues (N8-49) necessary for DNA binding. The small size of the N8-49 Killin peptide required for host cell lethality, and the incorporation of multiple cloning sites within its coding sequence through silent mutations, and allows for convenient and highly efficient cloning of any foreign DNA fragments, including PCR products using blunt-end ligation. The strong T5 promoter, under the lac operator control, located upstream of the toxic His-tagged Killin peptide also enables bacterial expression and convenient purification of any target gene cloned. This makes pKILLIN a truly versatile positive-selection cloning vector.

### BRIEF DESCRIPTION OF THE DRAWINGS

Table 1. Primers used in this study.

FIG. 1A. Structure of pKILLIN and its toxicity in E. coli. Plasmid map of pKILLIN with DNA sequence encoding the His-tagged Killin peptide and the MCS.

FIG. 1B. Structure of pKILLIN and its toxicity in E. coli. The toxicity of pKILLIN was assessed by transformation into XL1-Blue cell and selected on LB-Amp plates with and without IPTG induction. No visible colonies were observed on the plate with IPTG, in contrast to the plate without IPTG, where hundreds of colonies were obtained. XL1-Blue host cells transformed by pQE32 and the host cells alone served as controls.

FIG. 1C. Structure of pKILLIN and its toxicity in E. coli. The toxicity of pKILLIN was also assessed after transformation into DH5α cells (no transcription repression) and selected on LB-Amp plates without IPTG. No colonies were seen when transformed with pKILLIN, whereas the pQE32 vector and host cells alone served as positive and negative controls, respectively.

FIG. 2A. Cloning efficiency of the multiple cloning sites in pKILLIN. Visualization of the cloning efficiency of pKILLIN. The lacZα gene fragments with either blunt ends (PCR products without purification) or sticky ends (digested with BamH I, Bgl II, Kpn I, Pst I, and BamH I/HindIII), were cloned into Sma I (for PCR product) and the corresponding restriction sites, respectively, and plated on LB-Amp plates with X-gal. Blue colonies represented recombinants, whereas white colonies resulted from vector self-ligation after contaminating exonuclease-cutting of the cloning junctions.

FIG. 2B. Cloning efficiency of the multiple cloning sites in pKILLIN. The positive-selection cloning efficiency of the lacZα into various single and double cloning sites within the MCS of pKILLIN. The cloning efficiency was calculated as the ratio of the number of blue colonies over the total colony number (blue plus white) of DH5α on each LB-amp plate.

FIG. 2C. Cloning efficiency of the multiple cloning sites in pKILLIN. Colony PCR verification of the lacZα cloned as a PCR product into Sma I site of pKILLIN. 13 blue colonies were analyzed to contain the expected the lacZα with vector alone as control template.

FIG. 2D. Cloning efficiency of the multiple cloning sites in pKILLIN. The lacZα gene cloned as a 523 bp PCR fragment into Sma I site of pKILLIN was also verified by double digestion with BamH I and Kpn I.

FIG. 3A. The long blunt-end DNA fragments cloning efficiency of pKILLIN. Colony PCR analysis of the cloning efficiency of T4 DNA fragments (1.5 kb). 12 colonies were randomly picked from the LB-plate as the templates with the pKILLIN vector and gel-purified T4 fragments as control.

FIG. 3B. The long blunt-end DNA fragments cloning efficiency of pKILLIN. For the efficiency of the Taq DNA fragments (2.5 kb) cloning, 12 colonies were randomly picked from the LB-plate and analyzed by colony PCR with the pKILLIN vector and gel-purified Taq fragments as control.

FIG. 3C. The long blunt-end DNA fragments cloning efficiency of pKILLIN. The cloning efficiency analysis of endo180 DNA fragments (5.6 kb) cloning into pKILLIN vector. Plasmids were extracted from 12 colonies picked from the LB plate as templates of the PCR and the pKILLIN vector alone was negative control.

FIG. 4A. Positive-selection cloning and expression of the adk gene in XL1-Blue cells. Colony PCR analysis of the cloning efficiency of the adk gene from E. coli. 13 colonies were randomly picked from the LB-amp plate and analyzed, with pKILLIN vector alone as a negative control.

FIG. 4B. Positive-selection cloning and expression of the adk gene in XL1-Blue cells. Verification of pKILLIN-AK obtained from colony PCR by double digestion with BamH I and HindIII

FIG. 4C. Positive-selection cloning and expression of the adk gene in XL1-Blue cells. SDS-PAGE analysis of AK protein expression in XL1-Blue cells with or without IPTG induction. XL1-Blue cells alone or with pQE32 served as negative controls.

FIG. 4D. Positive-selection cloning and expression of the adk gene in XL1-Blue cells. AK activity levels of XL1-Blue alone and XL1-Blue containing either pQE32 or pKILLIN-AK with or without the introduction of IPTG.

FIG. 4E. Positive-selection cloning and expression of the adk gene in XL1-Blue cells. SDS-PAGE analysis of the His-tagged AK purification. The IPTG induced sample was eluted by a linear gradient of 0-500 mM imidazole in elution buffer and the fractions of Elution 4 and 5 containing His-AK were analyzed along with the induced sample and Ni-NTA column flow-through sample.

### DESCRIPTION OF SEQUENCE LISTING

SEQ ID NO. 1 Shows the amino acid sequence of the N-terminal region of minimal active Killin peptide sequence used in the pKillin vector for positive selection.

SEQ ID NO. 2 Shows the nucleotide coding sequence of SEQ ID NO. 1.

SEQ ID NO. 3 Shows the amino acid sequence of the N terminal His-tagged Killin peptide of SEQ ID NO. 1, a manmade peptide sequence expressed by the pKillin vector disclosed herein.

SEQ ID NO. 4 Shows the nucleotide coding sequence of SEQ ID NO. 3.

### DETAILED DESCRIPTION

The detailed description set forth below in connection with the appended drawings and sequence listing is intended as a description of presently preferred embodiments of the invention and does not represent the only forms in which the present invention may be constructed and/or utilized. The description sets forth the functions and the sequence of steps for constructing and operating the invention in connection with the illustrated embodiments. The term “Killin” as used herein refers to the Killin protein as taught in U.S. Pat. No. 7,576,191, incorporated herein by reference.

Construction of pKILLIN

In varying embodiments, one or a plurality of cloning sites may be incorporated into a 150 bp DNA template encoding Killin/N2-49 peptide, silent mutations were introduced via gene synthesis. The resulting Killin cassette was first cloned into pUC18 as an EcoR I-HindIII fragment. To create a His-tagged Killin/N2-49 cassette with multiple cloning sites, the above plasmid was used as a template for PCR amplification using primers L-killin-BamH I and R-killin-HindIII (Table 1) under the following PCR condition: 94° C., for 30 s, 57° C. for 20 s and 72° C. for 40 s, for 30 cycles, with Taq polymerase. After gel purification, the Killin/N8-49 encoding fragment was digested with BamH I and HindIII, purified by phenol/chloroform (3:1) extraction and ethanol precipitation, and ligated into the corresponding restriction sites in pQE32 (QIAGEN) using T4 DNA ligase (Takara). The resulting plasmid containing the His-tagged Killin/N2-49 with multiple cloning sites was transformed into XL1-Blue host cells, verified by DNA sequence analysis and named as pKILLIN.

Toxicity Test of pKILLIN

To test the toxicity of pKILLIN in E. coli, when induced by IPTG in the E. coli, 100 μL of XL1-Blue competent cells were mixed with either 100 ng of pKILLIN or pQE32 vectors. These cells were then incubated on the ice for 30 mM, heat-shocked at 42° C. for 90 s, immediately incubated on ice for 3 mM, 900 μL of Luria-Bertani (LB) medium without any antibiotics added, and then incubated at 37° C. with shaking at 220 rpm for 1 hr. 100 μL of the culture was then plated on LB-ampicillin (50 μg/mL) agar plates with or without IPTG (1 mM) respectively and incubated at 37° C. overnight. Similarly, pKILLIN or pQE32 were transformed into DH5α competent cells as described before, and plated on LB-ampicillin (LB-Amp) agar plates without IPTG.

Positive-Selection Cloning of PCR Amplified DNA Fragment into pKILLIN

The lacZα fragment (446 bp) from plasmid PCR-Trap-lacZ (anti tetracycline and no restriction sites inside lacZα) was PCR amplified with L-lac and R-lac primers (Table 1) under the following PCR conditions: 94° C. for 30 s, 55° C. for 20 s, 72° C. for 1 mM, for 30 cycles. Peptide 10 μL PCR product (diluted 1:5 to reduce the dNTPs if needed) directly ligated with 1 μL Phenol/chloroform purified Sma I-digested pKILLIN at 16° C. for 12-16 hr and followed by transformation into DH5α competent cells: 20 μL ligation products mixed with 200 μL DH5α competent cells, incubated on the ice for 1 hr, heat-shocked at 42° C. for 90 s, immediately incubated on ice for 3 min, 800 μL of Luria-Bertani (LB) medium without any antibiotics was added, and then incubated at 37° C. with shaking at 220 rpm for 1 hr, 4000 rpm centrifuged for 10 min, discard 900 μL supernatant and mixed with 5 μL IPTG (1M), then plated on LB-ampicillin (50 μg/mL) plates which was pre-plated with 50 μL X-gal (20 mg/mL) on the surface. Blue colonies containing the correct 446 bp lacZα fragments were verified by colony PCR with primers His-F and R-QE (Table 1) with the same PCR conditions described above. To further confirm lacZα was indeed inserted into Sma I site, recombinant plasmids were digested with BamH I and Kpn I, which flank the Sma I cloning site in pKILLIN.

Because of its high cloning efficiency, pKILLIN also can be used to clone the long PCR DNA fragments or even to construct cDNA libraries. To test the cloning efficiency of the long DNA fragments, T4 (1.5 kb) and Taq (2.5 kb) DNA fragments were amplified by Q5 high-fidelity DNA polymerase (NEB) with L-T4/R-T4 and L-Taq/R-Taq primers (Table 1) respectively under the following PCR conditions: 98° C. for 10 s, 57° C. for 20 s, 72° C. for 2 min, for 30 cycles. After gel purification, the PCR products were ligated with Sma I-digested pKILLIN and transformed into DH5α competent cells. 12 colonies of each plate were picked randomly and verified by colony PCR with His-F and R-QE primers (Table 1) under the conditions: 94° C. for 30 s, 55° C. for 20 s, 72° C. for 3 min, for 30 cycles. Furthermore, endo180 (5.6 kb) fragment was amplified with L-endo180 and R-endo180 primers (Table 1) under the conditions: 98° C. for 10 s, 57° C. for 20 s, 72° C. for 5 min, for 30 cycles. After gel purification, the endo180 fragments was ligated with pKILLIN and transformed into DH5α competent cells. We randomly restreaked 12 colonies on LB-ampicillin agar plates and extracted the plasmids respectively. Diluted to 1:200 as template, the plasmids were verified by PCR with primers His-F and R-QE by Q5 high-fidelity DNA polymerase under the same PCR conditions.

Positive-Selection Cloning of DNA Fragments at the Multiple Cloning Sites in pKILLIN Via Single or Double Restriction Digestions

To test the multiple cloning sites inside the (N8-49) gene, primers were designed to amplify the lacZα fragments with different restriction sites located at the ends of the PCR products (Table 1). After gel purification, the PCR products were digested with either single (BamH I, Kpn I, Pst I, Bgl II) or double restriction enzymes (BamH I and HindIII), and then ligated into corresponding restriction sites in pKILLIN. Ligation products were transformed into DH5α and plated on LB-Amp plates and scored for lacZα via blue color as described before.

### Positive-Selection Cloning, Expression and Purification

The Adenylate kinase (adk) gene from E. coli (22), was amplified as a 664 bp PCR fragment from colony lysate using E. coli K12 genomic DNA as a template and PCR primers L-AK-BamH I and R-AK-HindIII (Table 1). PCR conditions used for the adk amplification were: 94° C. for 30 s, 57° C. for 20 s and 72° C. for 1 min, for 30 cycles. Following gel purification, the adk fragment was digested with BamH I and HindIII, purified by phenol/chloroform (3:1) extraction and ligated into the corresponding restriction sites in pKILLIN to allow adenylate kinase (AK) to be expressed as a His-tagged fusion protein. After transformation into XL1-Blue and selection on LB plates with ampicillin (50 μg/mL) and IPTG (1 mM), recombinant plasmids were verified by both restriction digestions (BamH I and HindIII) and colony PCR using His-F and R-QE primers (Table 1) with PCR conditions: 94° C. for 30 s, 57° C. for 20 s and 72° C. for 40 s, for 30 cycles. A single colony containing pKILLIN-AK was verified for the expression of His-tagged AK IPTG induction using 12% SDS-PAGE and Coomassie Blue staining. In addition, an AK enzyme activity assay was performed to confirm the expression following the method described previously (23). IPTG induced sample with His-tagged AK was obtained as described before and dissolved in binding buffer (50 mM Na3PO4, 500 mM NaCl, pH 7.2). The sample was applied to the pre-equilibrated HisTrap FF column (GE Healthcare) filled with 1 ml Ni Sepharose medium, washed with binding buffer at 1 ml/min and eluted by a linear gradient of 0-500 mM imidazole in elution buffer (50 mM Na3PO4, 500 mM NaCl, pH 7.2) at 1 ml/min IPTG induced sample, flow-through and the eluted fractions were analyzed by 15% SDS-PAGE and Coomassie Blue staining.

### Results

Construction of Positive-Selection Cloning Vector pKILLIN

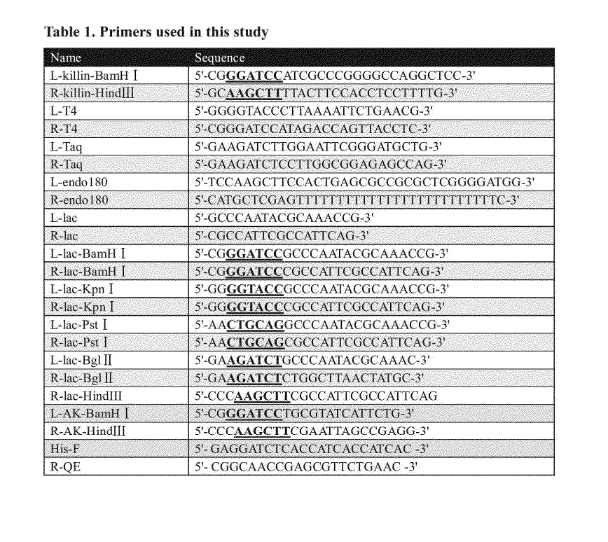

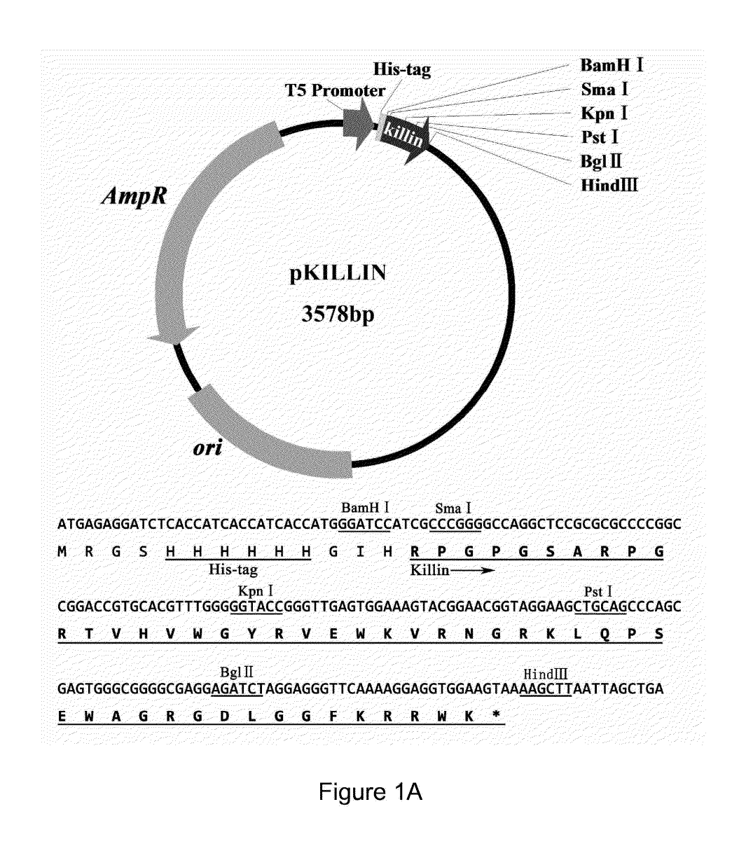

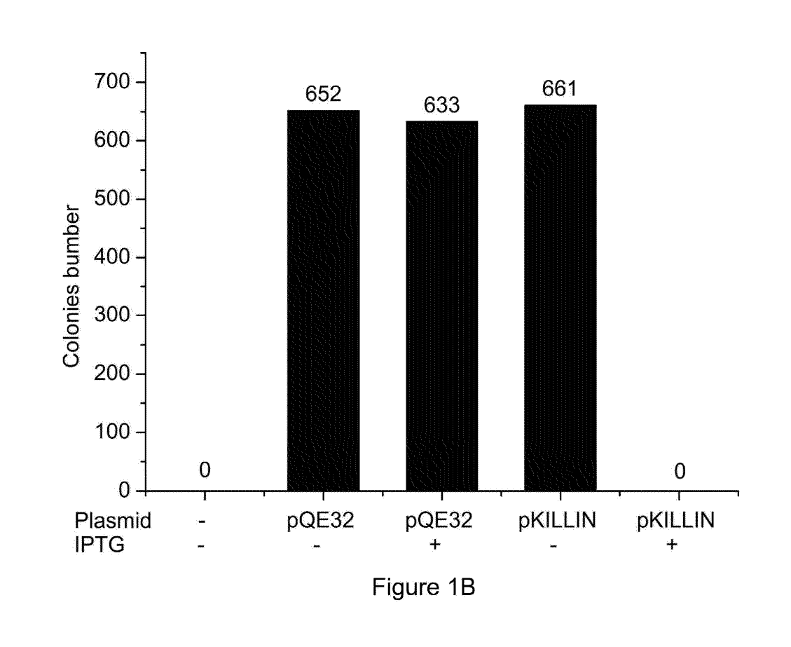

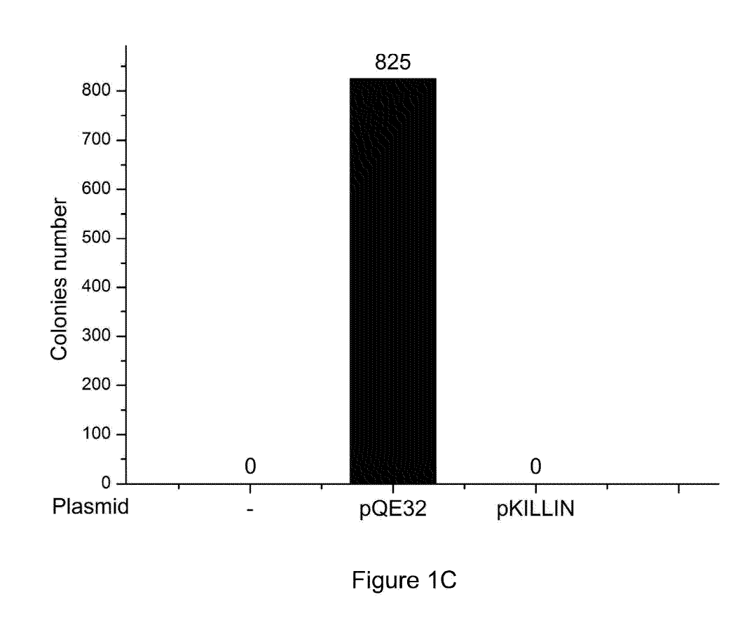

A truly useful positive-selection cloning vector ideally should have the following attributes: (1) low self-ligation background; (2) contain multiple cloning sites with commonly used restriction enzymes; (3) be able to clone PCR products directly in addition to restriction fragments; (4) be able to express the cloned gene of interest; (5) be able to purify the protein expressed. In order to achieve these goals, we took advantage of the minimal 42 aa DNA binding domain of Killin responsible for toxicity in E. coli. We used gene synthesis to introduce the desired multiple cloning sites into the killin sequence via silent mutations. The resulting killin fragment was cloned in-frame with the His-tag in the pQE32 expression vector between the HindIII and BamH I sites. The resulting plasmid was confirmed by DNA sequence analysis and dubbed pKILLIN vector (FIG. 1A). The multiple cloning sites included six commonly used restriction endonuclease sites that are unique sites in pKILLIN: BamH I, Sma I, Kpn I, Pst I, Bgl II and HindIII. Under the control of a strong T5 promoter and the lac operator, the 42 aa Killin/N8-49 peptide may be expressed as a His-tagged fusion protein (FIG. 1A).

To test the lethality of the His-tagged Killin peptide, pKILLIN vector was first transformed into XL1-Blue host cells, which contain the lacI^{q} mutation, and plated on the LB-Amp plates in the presence and absence of IPTG. As predicted, no colonies formed on the plate with IPTG induction, whereas hundreds of colonies were obtained when the promoter driving the His-tagged Killin peptide was repressed in the absence of IPTG (FIG. 1B). As controls, pQE32 vector gave similar number of transformants whether IPTG was added or not, and the host cells alone gave no Amp-resistant colonies. Concurring results were obtained when pKILLIN and the pQE32 control vector were transformed into DH5α host cells. These do not contain the lacI^{q} mutation, thus exhibiting incomplete repression of the His-tagged Killin peptide in the absence of IPTG (FIG. 1C). These results clearly established the total lethality of pKILLIN in E. coli due to the expression of the His-tagged Killin peptide. This also ensures a clear background in cases of vector self-ligation due to the powerful positive-selection pressure of Killin in E. coli.

### Positive-Selection Cloning of Foreign DNA Fragments

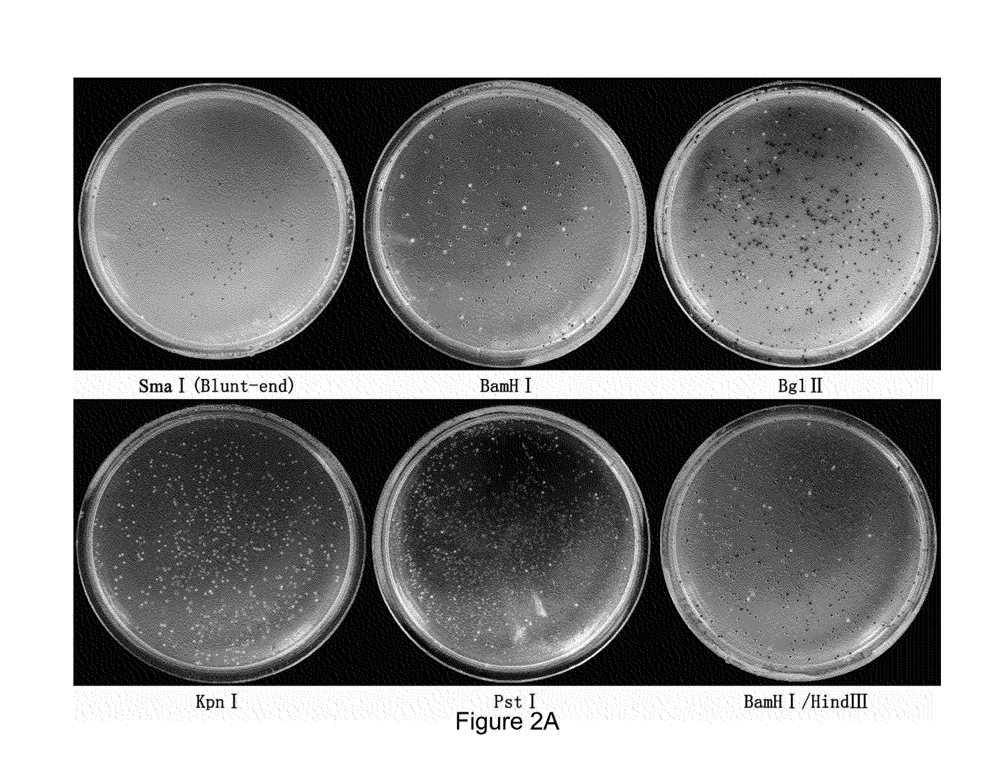

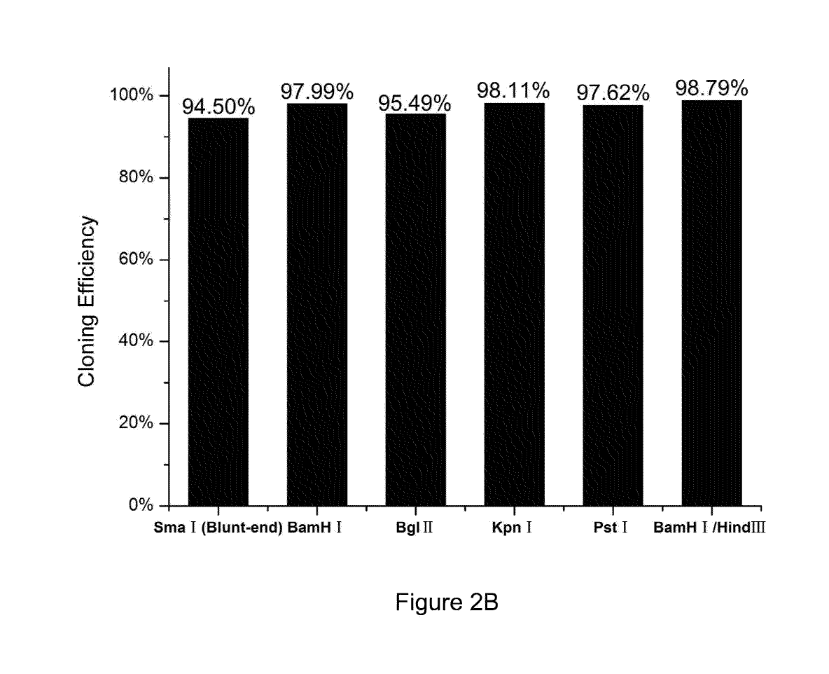

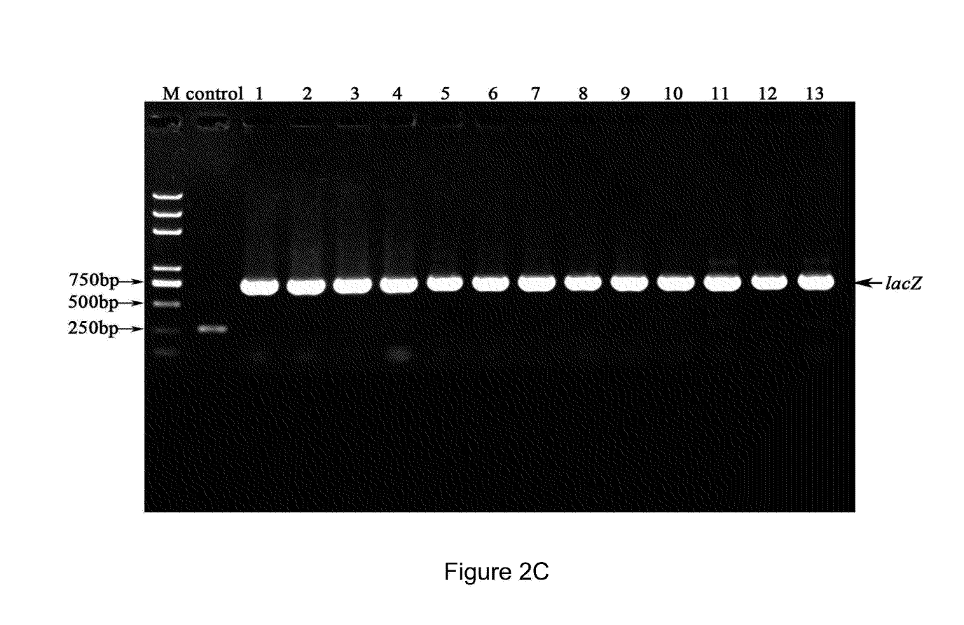

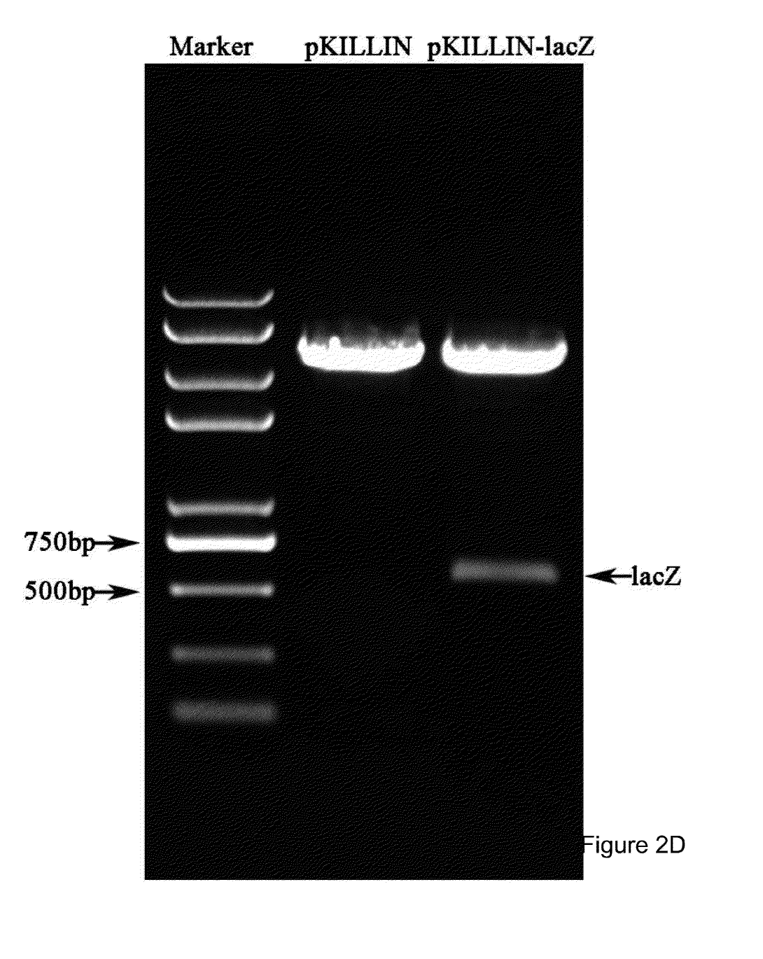

First, we tested if the pKILLIN vector could allow direct cloning of PCR products via positive-selection. To do so, with consideration of an accurate quantification of the cloning efficiency, we PCR amplified the 446 bp DNA template encoding the lacZα peptide and directly ligated it without any purification into the Sma I site of pKILLIN. After transformation into DH5α, the transformants were plated on LB-Amp plates containing X-gal and IPTG. The resulting plate showed that most of the colonies were blue in color, consistent with the successful cloning of the lacZα gene (FIG. 2A). Subsequent statistical analysis of the colony-PCR assay as well as the restriction digestions showed that the cloning efficiency of the positive-selection was 95% (FIG. 2B) and all of the blue colonies carried the correct lacZα recombinant (FIG. 2C, D). Similarly, insertion of the PCR amplified lacZα into pKILLIN after single restriction digestion with BamH I, Sma I, Kpn I, Pst I, Bgl II or double digestions with BamH I and HindIII also generated mostly blue colonies (FIG. 2A) with a cloning efficiency of 95-99% (FIG. 2B).

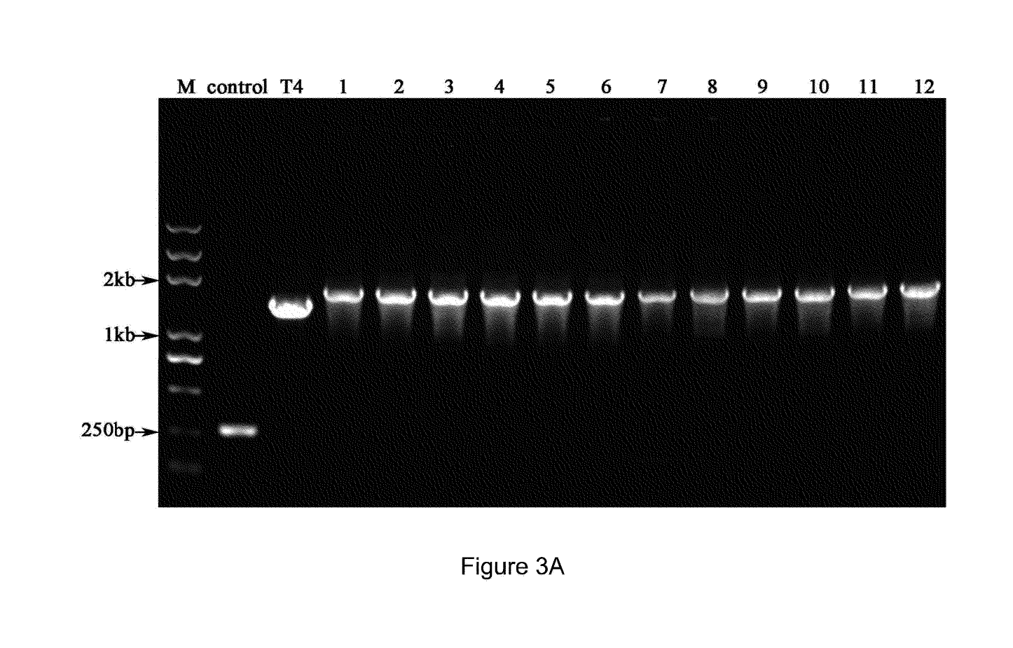

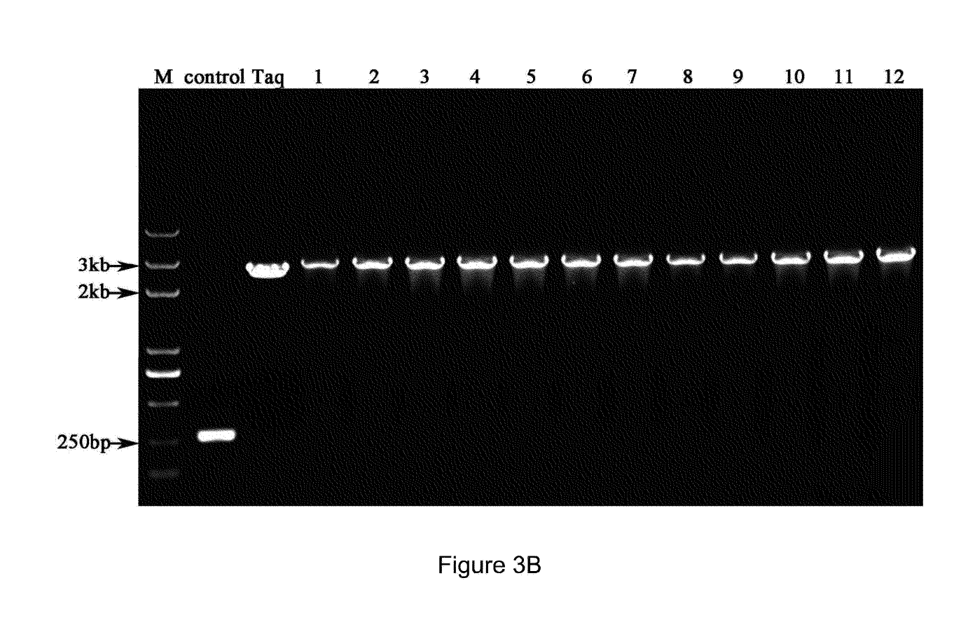

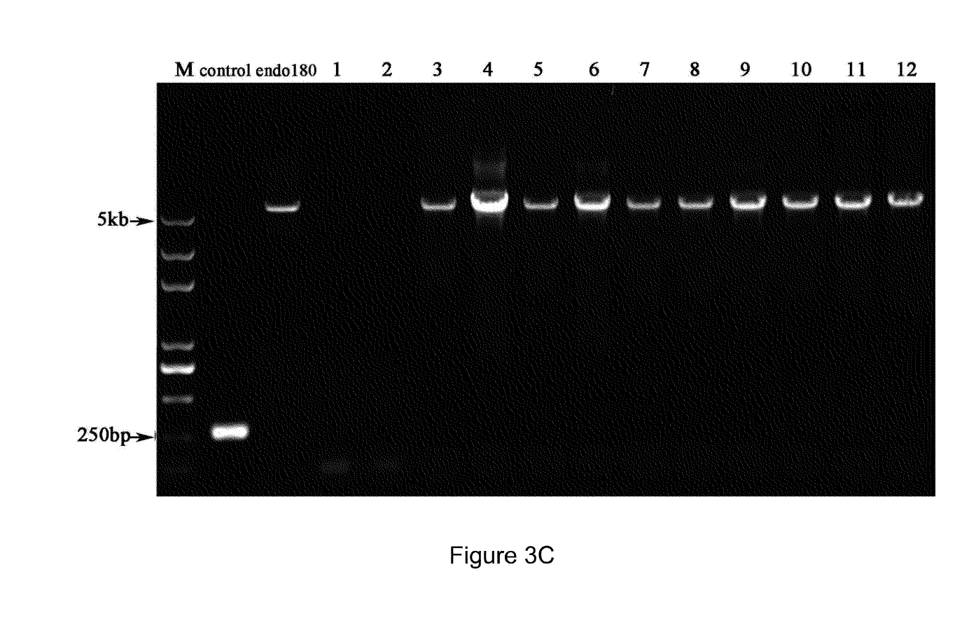

For the long blunt-end DNA fragments, T4 (1.5 kb), Taq (2.5 kb) and endo180 (5.6 kb) were cloned into Sma I site of pKILLIN and the colony PCR analysis showed that the cloning efficiency for 1.5 kb or 2.5 kb DNA fragments were almost 100% (all the 12 clones each assay containing complete PCR products), and the efficiency of 5.6 kb fragments were even almost 80% (10 of 12 colonies containing complete PCR products), this suggested that pKILLIN vector was perfect for the long DNA fragments cloning (FIGS. 3A-C).

### Positive-Selection Cloning and Expression of the Adk Gene

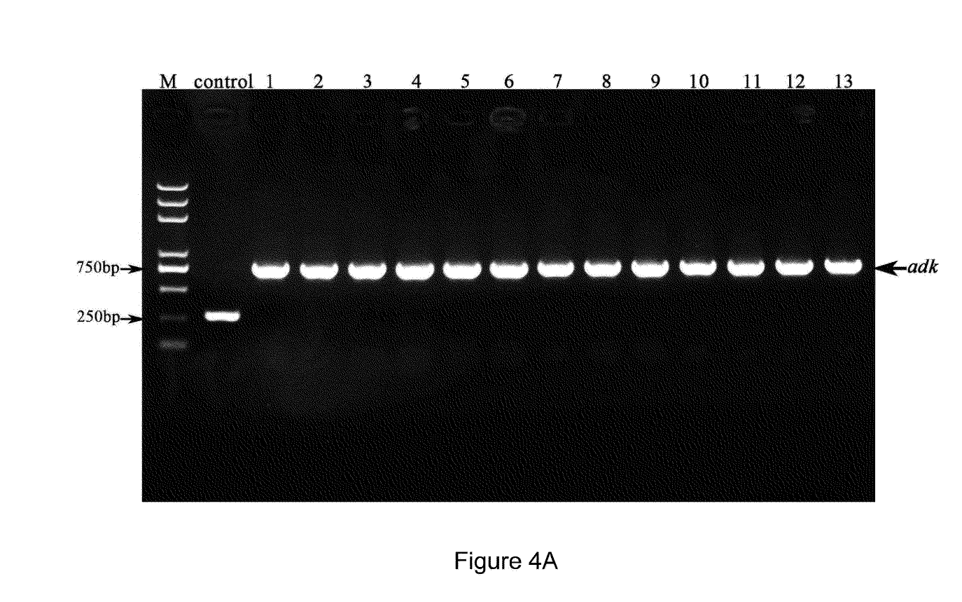

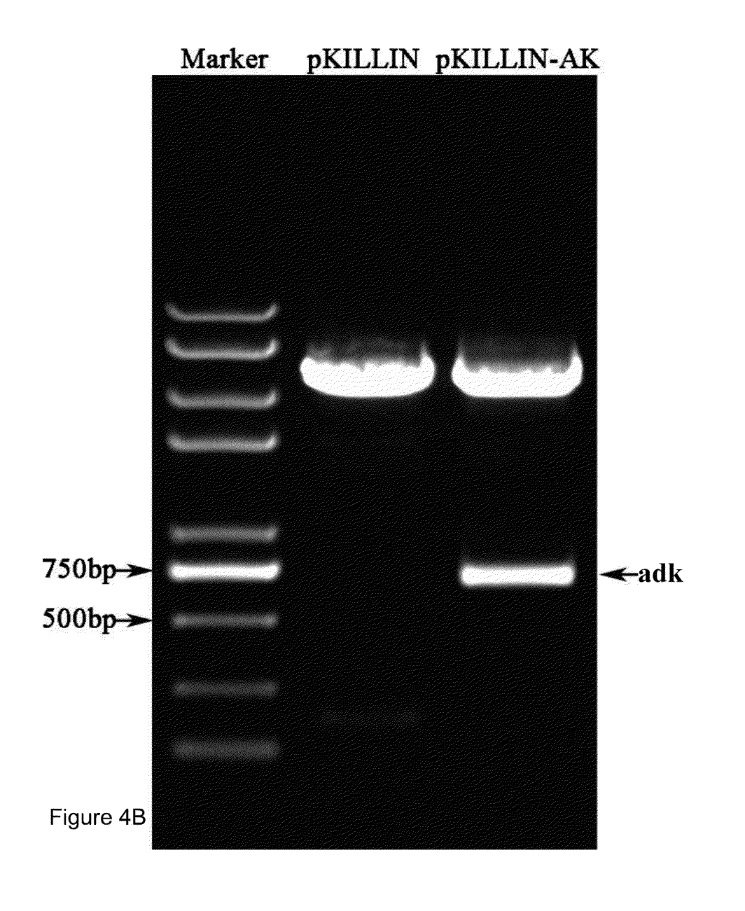

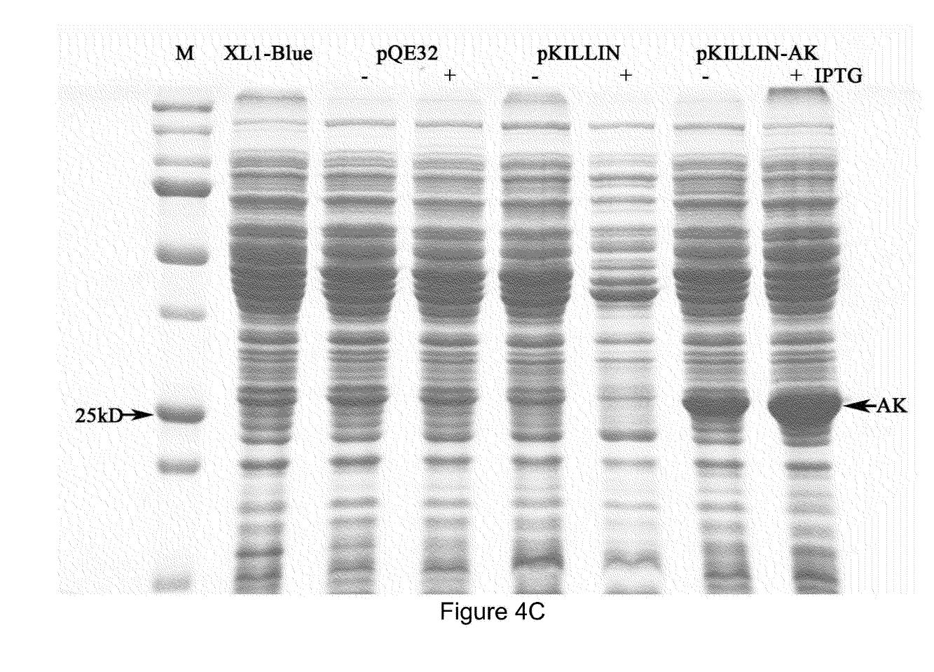

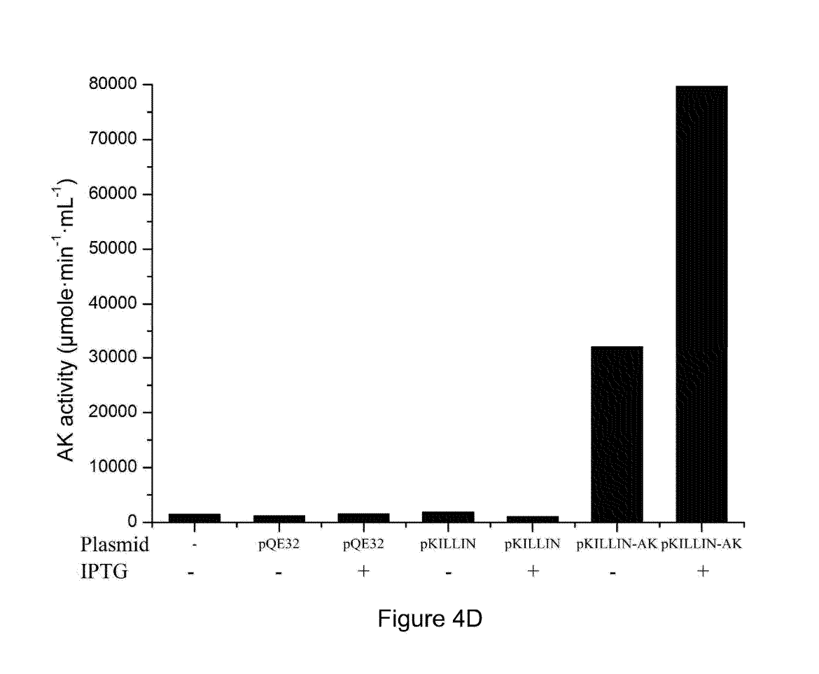

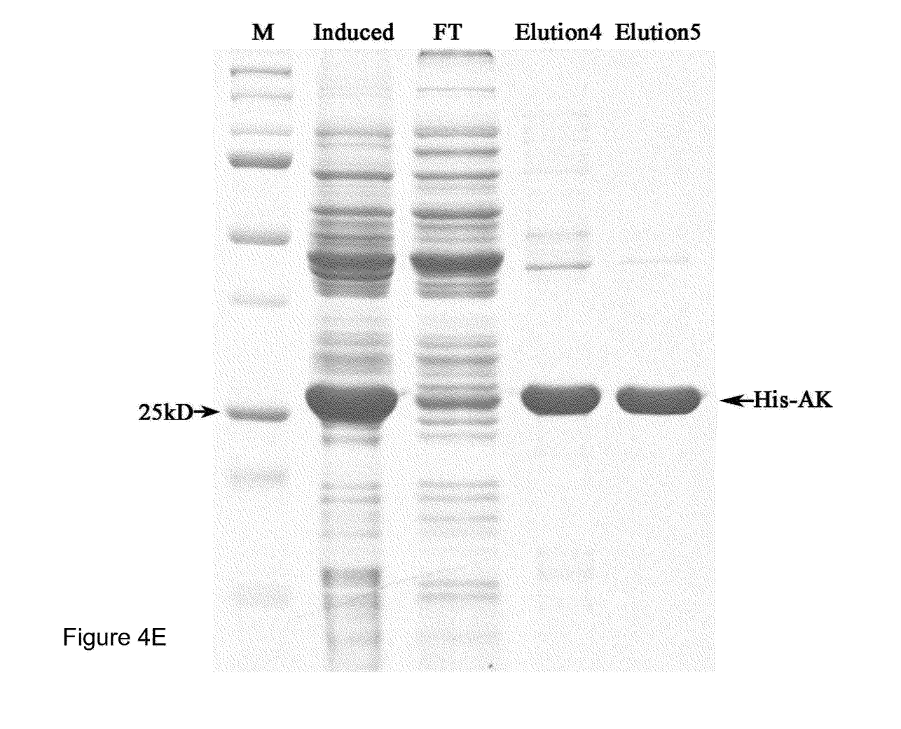

To test the ability of positive-selection cloning and direct expression of a foreign gene in pKILLIN, the adk gene from E. coli was PCR amplified, digested and in-frame cloned with the His-tag as a BamH I and HindIII fragment into pKILLIN and transformed into XL1-Blue with IPTG induction. Randomly picked colonies were checked by colony PCR assay for the adk insert. The expected adk band (772 bp) appeared in all 13 colonies analyzed, and the resulting plasmid was named pKILLIN-AK (FIG. 4A). Digestion by BamH I and HindIII as well as DNA sequencing analysis also confirmed that pKILLIN-AK plasmid had the correct insert (FIG. 4B). XL1-Blue cells carrying the pKILLIN-AK plasmid were cultured in LB broth in the presence and absence of IPTG. SDS-PAGE analysis of protein extracts showed a high level expression of the 27 Kda His-tagged adenylate kinase (AK) with IPTG induction in comparison with the host cell alone or vector controls (FIG. 4C). Subsequent enzyme activity assay confirmed that pKILLIN-AK containing XL1-Blue cells had a much higher adenylate kinase activity than that from the host cells alone or cells carrying the pQE32 control vector, whether IPTG was present or not (FIG. 4D). For the purification test, almost all the His-tagged AK in the induced sample was eluted with high purity, which showed that pKILLIN was prominent for the His-tag purification (FIG. 4E).

### Discussion

The pKILLIN vector utilizes a truncated killin gene encoding the 49 aa N2-49 DNA binding domain to confer total lethality in E. coli. The minimal size (150 bp) of the indicator gene essential for lethality and the multiple commonly used restriction sites built-in through silent mutations via gene synthesis (FIG. 1A) ensure both convenient and highly efficient positive-selection cloning with minimal background false positive colonies. Our results indicate that pKILLIN can be propagated in E. coli hosts with lacI^{q} genotype, such as XL1-Blue in which the expression of the Killin peptide is under the control of the lac operator. However, when either induced by IPTG or in lacI wild type hosts, such as DH5α in which the expression of killin cannot be inhibited because of the high copy numbers of the vector, pKILLIN conferred total lethality to the host cells with zero background from vector self-ligation (FIG. 1B, C). The extreme toxicity of the Killin peptide in E. coli is likely mediated by its ability to interrupt DNA replication due to its high affinity to single-stranded DNA, as seen in mammalian cells. The potency of Killin in conferring lethality in E. coli could be judged by its barely detectable expression level when de-repressed on SDS-PAGE or through Western blot analysis (data not shown). The use of pKILLIN in positive-selection cloning requires no special bacterial hosts or additional substrates.

The efficacy for highly efficient positive-selection cloning was first demonstrated in direct cloning of PCR products through blunt-end ligation into the unique Sma I site in pKILLIN (FIG. 2). In the past, direct cloning of PCR products is often inefficient and mainly achieved with either TA cloning vectors or vectors with uncommon restriction endonucleases. Unlike most of the previous work, we selected the traceable lacZα as the tester gene for accurate scoring for the cloning efficiency. The PCR-amplified lacZα fragment, without any post-PCR purification, was easily cloned into pKILLIN. The true cloning efficiency reached 95%, as tabulated by the number of blue colonies divided by total colony numbers (FIG. 2B). The cloning efficiency is even better after gel purification of the PCR products (data not shown). DNA sequence analysis revealed that the existence of a few white colonies on the plates was attributed to the presence of exonucleases, often found as an impurity in either the T4 DNA ligase or restriction endonucleases used to cut the vector. Star activity under non-optimal conditions, which resulted in frame-shift mutations in killin at the cloning site from self-ligated vectors (data not shown) may also be a cause. In fact, this finding may allow pKILLIN to be used as a simple and inexpensive assay for the detection and quantification of exonuclease contaminations (e.g. in commercial T4 DNA ligase and restriction enzymes). Besides highly efficient cloning of PCR products, pKILLIN may be very useful as a shuttling vector in which PCR amplified DNA fragments of interest with restriction sites designed at both ends can be first directly cloned into the Sma I site, then subsequently cut out with high efficiency for subcloning into the final targeting vectors. This would alleviate the problem often encountered with cutting restriction sites located at the extreme ends of DNA fragments, which dramatically lowers the downstream cloning efficiency. In addition to cloning of PCR products, the inclusion of multiple cloning sites in pKILLIN also allows for the choice of several commonly used restriction sites to be employed for positive-selection cloning. These sites have been successfully demonstrated with 96-99% cloning efficiency in cloning the lacZα fragment cut with either single or double restriction enzymes (FIG. 2A, B). The few background white colonies seemed to arise from reasons listed above.

With the high efficiency in positive-selection cloning, pKILLIN is also able to directly express and purify any foreign gene with His-tag fusion in the N-terminus. The T5 promoter is a strong transcription initiator and widely used in Gram-negative or Gram-positive organisms. Unlike the T7 expression system which requires hosts with the λ derivative DE3 integrated into the chromosome, foreign genes can be expressed with pKILLIN in any host bacteria. In this study, we demonstrated that when cloned into pKILLIN, the His-tag fused adenylate kinase from E. coli became highly expressed and efficiently purified with IPTG induction in XL1-Blue cells (FIG. 4E). The visible leaky expression of AK without IPTG induction could be attributed to the high copy number of pKILLIN and the strong T5 promoter (FIG. 4C, D).

In conclusion, here we presented a multi-purpose positive-selection cloning vector based on the toxicity of killin in E. coli. Due to the small size of the Killin/N2-49 peptide that is essential for the extreme toxicity in bacteria, in comparison with any previously employed indicator genes in positive-selection cloning vectors, pKILLIN is the most versatile and efficient positive-selection cloning vector ever created. It can not only efficiently clone any DNA fragments, including PCR products directly without any purification, but also express any gene of interest cloned at high level in E. coli for subsequent purification as a His-tagged fusion protein. Besides, serving as a cloning trap, pKILLIN may be used to create genomic or cDNA libraries. Given the high cloning efficiency demonstrated in this report, positive-selection cloning with pKILLIIN should offer a great alternative to cloning vectors employing blue/white selection.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

1. A positive-selection cloning vector capable of transforming a prokaryotic cell, said cloning vector comprising an inducible promoter linked to a template encoding the toxic gene, killin in which a multiple cloning site is introduced via silent DNA mutations, said cloning vector is maintained and propagated in a prokaryotic host under non-inducible condition.

2. The positive-selection cloning vector of claim 1 wherein. The positive-selection cloning vector is a recombinant plasmid.

3. The positive-selection cloning vector of claim 1 wherein the inducible promoter is a lac promoter.

4. The positive-selection cloning vector of claim 1 wherein the inducible promoter is a lac promoter.

5. The positive-selection cloning vector of claim 1 wherein the killin gene of encodes a minimal domain of KILLIN protein essential to confer lethality to the prokaryotic host upon expression.

6. The positive-selection cloning vector of claim 5 wherein the minimal domain of KILLIN protein has an amino acid sequence of SEQ ID NO. 1.

7. The positive-selection cloning vector of claim 6 wherein a DNA template encoding the minimal domain of KILLIN protein has a nucleotide sequence shown as SEQ ID NO. 2.

8. The positive-selection cloning vector of claim 1 wherein the killin gene is in-frame fused to an affinity tag.

9. The positive-selection cloning vector of claim 8 wherein the affinity tagged KILLIN has an amino acid sequence of SEQ ID NO. 3.

10. The positive-selection cloning vector of claim 9 wherein a DNA template encoding the affinity tagged KILLIN has a nucleotide sequence of SEQ ID NO. 4.

11. The positive-selection cloning vector of claim 1 wherein the multiple cloning site contains at least one restriction site.

12. The positive-selection cloning vector of claim 11 wherein the multiple cloning site contains a Sma I restriction site.

13. The positive-selection cloning vector of claim 1 wherein the multiple cloning site contains more than one restriction site.

14. The positive-selection cloning vector of claim 13 wherein the multiple cloning site contains BamHI, SmaI, KpnI, PstI, BglII and HindIII restriction sites.

15. The positive-selection cloning vector of claim 1 wherein the prokaryotic host is Escherichia coli.

16. The positive-selection cloning vector of claim 1 wherein the prokaryotic host is Escherichia coli host XL-1 Blue.

17. The positive-selection cloning vector of claim 1 wherein the non-inducible condition is without adding IPTG into a culture medium.

18. The positive-selection cloning vector of claim 1 wherein the positive-selection cloning vector is a recombinant virus.

19. A method for selecting recombinants comprising:
(a) ligating a DNA fragment into any restriction site within the multiple cloning site of the cloning vector according to claim 1, which leads to inactivation of the killin, thus conferring to recombinant vectors an ability to form colonies upon transformation into a bacterial host under inducible condition or without repression; whereas a non-recombinant vector expressing the killin gene is toxic to the host cell making them unable to form colonies.
(b) propagating the recombinants for further characterizations, comprising a DNA sequence analysis, a subcloning, and a protein expression.

20. The method of claim 19 wherein the DNA fragment is blunt-ended.

21. The method of claim 19 wherein the DNA fragment is sticky-ended.

22. The method of claim 19 wherein the DNA fragment is produced by polymerase chain reaction.

23. The method of claim 19 wherein the DNA fragment is genomic DNA.

24. The method of claim 19 wherein the DNA fragment is a cDNA.

25. The method of claim 19 wherein the inducible condition is adding IPTG into the culture medium.

26. The method of claim 19 wherein a bacterial host without repression is the Escherichia coli host DH5-alpha.


SEQUENCE LISTING (4 sequences)

SEQ ID NO: 1 - protein, 49 aa, Homo sapiens
RPGPGSARPGRTVHVWGYRVEWKVRNGRKLQPSEWAGRGDLGGFKRRWK

SEQ ID NO: 2 - DNA, 150 bp, Artificial Sequence
  Formed via gene synthesis
cgcccggggccaggctccgcgcgccccggccggaccgtgcacgtttgggggtaccgggttgagtggaaagtacggaacggtaggaagctgcagcccagcgagtgggcggggcgaggagatctaggagggttcaaaaggaggtggaagtaa

SEQ ID NO: 3 - protein, 62 aa, Artificial Sequence
  Manmade peptide sequence
MRGSHHHHHHGIHRPGPGSARPGRTVHVWGYRVEWKVRNGRKLQPSEWAGRGDLGGFKRRWK

SEQ ID NO: 4 - DNA, 189 bp, Artificial Sequence
  Formed via gene synthesis
atgagaggatctcaccatcaccatcaccatgggatccatcgcccggggccaggctccgcgcgccccggccggaccgtgcacgtttgggggtaccgggttgagtggaaagtacggaacggtaggaagctgcagcccagcgagtgggcggggcgaggagatctaggagggttcaaaaggaggtggaagtaa